(19) United States
(12) Patent Application Publication
(10) Pub. No.: US 2024/0185418 A1
(43) Pub. Date: Jun. 6, 2024

(54) SYSTEM AND METHOD FOR INTRAORAL IDENTIFICATION

(71) Applicant: GET-GRIN INC., Airmont, NY (US)

(72) Inventor: Yarden EILAT-BLOCH, Haifa (IL)

(21) Appl. No.: 18/439,040

(22) Filed: Feb. 12, 2024

Related U.S. Application Data
(63) Continuation of application No. PCT/US22/40265, filed on Aug. 12, 2022.
(60) Provisional application No. 63/233,205, filed on Aug. 14, 2021.

(51) Int. Cl. G06T 7/00; A61C 9/00; G06T 7/90

(52) U.S. Cl. CPC G06T 7/0014; A61C 9/0053; G06T 7/90; G06T 2207/10024; G06T 2207/30036

(57) ABSTRACT

A color reference target for intraoral imaging, comprising one or more interchangeable color samples, wherein the color reference target is configured to be coupled to an intraoral adapter comprising a viewing channel configured to define a field of view of an intraoral region of a subject's mouth, and wherein the color reference target appears in the field of view of the subject's mouth.

### CROSS-REFERENCE TO RELATED APPLICATIONS

The present application is a Continuation application of International Patent Application PCT/US22/40265, filed on Aug. 12, 2022, which claims the benefit of U.S. Provisional Application Ser. No. 63/233,205, filed Aug. 14, 2021, each of which is incorporated herein by reference in its entirety.

### TECHNICAL FIELD

The present invention, in at least some embodiments, relates to a method and a system for dental imaging, and more specifically a method and system for identifying the color or shade of teeth or other intraoral landmarks.

### BACKGROUND

Dental professionals and orthodontists may treat and monitor a user's dental condition based on in-person visits. Treatment and monitoring of a user's dental condition may require a user to schedule multiple in-person visits to a dentist or orthodontist. The quality of treatment and the accuracy of monitoring may vary depending on how often and how consistently a user sees a dentist or orthodontist. In some cases, suboptimal treatment outcomes may result if a user is unable or unwilling to schedule regular visits to a dentist or orthodontist.

### SUMMARY

Recognized herein is a need for remote dental monitoring solutions to allow dental users to receive high quality dental care, without requiring a dental professional to be physically present with the user. Some dental professionals and orthodontists may use conventional teledentistry solutions to accommodate users' needs and schedules. However, such conventional teledentistry solutions may provide inadequate levels of supervision. Further, such conventional teledentistry solutions may be limited by an inaccurate or insufficient monitoring of a user's dental condition based on one or more photos taken by the user, if the photos do not adequately capture various intraoral features.

The present disclosure provides methods and systems for intraoral imaging with a capability to identify the color or shade of teeth or other intraoral landmarks by using a color reference target to enhance remote dental monitoring capabilities. As used herein, intraoral imaging may refer to the acquisition of one or more intraoral videos and/or intraoral images. The methods and systems disclosed herein may provide a convenient solution and user experience for dental users to capture one or more intraoral videos or images using a mobile device such as a smartphone. The methods and systems disclosed herein may provide dentists and orthodontists with a detailed analysis of the user's dental condition based on one or more dental scans captured remotely by the user.

In some cases, when a dental user takes an intraoral image or video, the colors in the image or video may appear different depending on the camera or mobile device which with the images or video were taken. This may present a difficulty in diagnosing medical conditions. For example, if a user takes a first intraoral image with an old cell phone camera and then takes a second intraoral image at a later date with a new cell phone camera, the colors in the images may appear different. This can make it hard to determine the clinical progression between the two images. The systems and methods described herein may allow the dental user or dental professional to view accurate colors of the intraoral images, thereby resulting in more accurate diagnoses.

In an aspect provided herein is a color reference target for intraoral imaging, comprising one or more interchangeable color samples, wherein the color reference target is configured to be visible when viewed through the viewing channel of an intraoral adapter, wherein the viewing channel of the intraoral adapter is configured to define a field of view of an intraoral region of a subject's mouth.

In some cases, the one or more color samples are based on a color quantification scheme. In some cases, the color quantification scheme is the Munsell color system. In some cases, at least one color sample of the one or more color samples corresponds to a color of a reference oral landmark. In some cases, the reference oral landmark comprises a color of a natural tooth. In some cases, the adapter is configured to couple to a camera configured to capture images or videos of the subject's mouth, wherein the captured images or videos comprise an oral landmark of the subject.

In some cases, the images or videos are captured under light at a wavelength of 390 nm to 450 nm. In some cases, the images or videos are captured under quantitative light induced fluorescence. In some cases, the images or videos are captured under light at a wavelength of 10 nm to 400 nm. In some cases, the images or videos are captured under ultraviolet light. In some cases, the images or videos are captured under light at a wavelength of 780 nm to 1,000,000 nm. In some cases, the images or videos are captured under infrared light.

In some cases, at least one of the one or more color samples corresponds to a color of a natural tooth, a color of an intraoral tissue under fluorescent light, a color of an intraoral tissue under ultraviolet light, or a color of an intraoral tissue under infrared light, or any combination thereof. In some cases, at least one color sample of the one or more color samples is interchangeable based on the color of an oral landmark of the subject. In some cases, the one or more color samples have the same Bidirectional Reflectance Distribution Function (BRDF) as an oral landmark of the subject.

In some cases, at least part of the color reference target can be covered with a material such that the color reference target has the same optical properties as a color reference target covered in saliva. In some cases, the one or more color samples can be interchanged manually. In some cases, the color reference target comprises a graphical display. In some cases, the color reference target is made from paper, cardboard, plastic, metal, porcelain, dentin or enamel, or a combination thereof. In some cases, the color reference target is configured to be coupled to the intraoral adapter. In some cases, the color reference target is configured to be coupled to an intraoral region of the subject's mouth. In some cases, at least one color sample of the one or more color samples is interchangeable based at least in part on the color or shade of an oral landmark of the subject.

In another aspect, provided herein is a method to determine the color, shade, or condition of an oral landmark of a subject by using the color reference target of claim 1 and a mobile device camera. In some cases, the mobile device is a smartphone.

In another aspect, provided herein is a system for identifying the color or shade of an oral landmark of a subject, comprising: an adapter comprising a viewing channel, wherein the viewing channel is configured to define a field of view of an intraoral region of a subject's mouth; and a color reference target comprising three or more color samples, wherein the color reference target appears in the field of view of the subject's mouth.

In some cases, the adapter is configured to couple to a camera configured to capture images or video of an oral landmark of the subject. In some cases, the camera is part of a mobile device. In some cases, the three or more color samples are based on a color quantification scheme. In some cases, the color quantification scheme is the Munsell color system. In some cases, at least one color sample of the three or more color samples corresponds to a color of a reference oral landmark. In some cases, the reference oral landmark comprises a color of a natural tooth.

In some cases, the images or videos are captured under light at a wavelength of 390 nm to 450 nm. In some cases, the images or videos are captured under quantitative light induced fluorescence. In some cases, the images or videos are captured under light at a wavelength of 10 nm to 400 nm. In some cases, the images or videos are captured under ultraviolet light. In some cases, the images or videos are captured under light at a wavelength of 780 nm to 1,000,000 nm.

In some cases, the images or videos are captured under infrared light. In some cases, the three or more color samples have the same Bidirectional Reflectance Distribution Function (BRDF) as an oral landmark of the subject. In some cases, at least part of the color reference target can be covered with a material such that the color reference target has the same optical properties as a color reference target covered in saliva. In some cases, at least one of the three or more color samples can be interchanged manually. In some cases, the color reference target comprises a display. In some cases, the color reference target is made from paper, cardboard, plastic, metal, porcelain, dentin or enamel, or a combination thereof. In some cases, at least one of the three or more color samples is interchangeable based on the color of an oral landmark of a subject. In some cases, a user changes the at least one of the three or more color samples using a mobile device.

In some cases, the color reference target is located on the adapter. In some cases, color reference target is located on the intraoral region of the subject's mouth. In some cases, the camera is configured to capture videos or images in color. In some cases, the camera is configured to capture videos or images in black and white. In some cases, the method described herein further comprises a processing unit, and wherein the processing unit processes a color of at least part of the oral landmark captured in the image or video to identify a true color of the at least part of the oral landmark, wherein the processing is based on the three or more color samples of the color reference target. In some cases, the oral landmark comprises at least part of a tooth of the subject. In some cases, the oral landmark comprises at least part of an oral tissue of the subject.

In some cases, the adapter comprises a flange that is configured to be coupled to the subject's mouth. In some cases, the adapter is configured to block environmental light from the field of view of the intraoral region of the subject's mouth. In some cases, the system further comprises a light source, and wherein the light source is configured to illuminate at least part of the viewing channel. In some cases, the light source is a mobile device. In some cases, the adapter is configured to be moved in the subject's mouth to adjust the field of view of the intraoral region of the subject's mouth.

In another aspect, provided herein is a method for identifying a true color of an oral landmark of a subject, comprising: (a) providing an adapter comprising a viewing channel configured to define a field of view of an intraoral region of the subject's mouth, wherein the oral landmark is visible in the field of view of the subject's mouth; (b) providing a color reference target comprising three or more color samples, wherein the color reference target appears in the field of view of the subject's mouth; (c) capturing videos or images of at least part of the oral landmark through the adapter viewing channel; and (d) using the color reference target, processing a color of at least part of the oral landmark captured in the videos or images to identify the true color of the at least part of the oral landmark, wherein the processing is based on the three or more color samples of the color reference target.

In some cases, the color reference target is located on the adapter. In some cases, the color reference target is located on the intraoral region of the subject's mouth. In some cases, the three or more color samples are based on a color quantification scheme. In some cases, the color quantification scheme is the Munsell color system. In some cases, at least one of the three or more color samples corresponds to a color of a reference oral landmark. In some cases, the reference oral landmark comprises a color of a natural tooth.

In some cases, the images or videos are captured under light at a wavelength of 390 nm to 450 nm. In some cases, the images or videos are captured under quantitative light induced fluorescence. In some cases, the images or videos are captured under light at a wavelength of 10 nm to 400 nm. In some cases, the wherein the images or videos are captured under ultraviolet light. In some cases, the images or videos are captured under light at a wavelength of 780 nm to 1,000,000 nm.

In some cases, the images or videos are captured under infrared light. In some cases, at least one of the three or more color samples corresponds to a color of a natural tooth, a color of an intraoral tissue under fluorescent light, a color of an intraoral tissue under ultraviolet light, or a color of an intraoral tissue under infrared light, or any combination thereof. In some cases, at least one of the three or more color samples has the same Bidirectional Reflectance Distribution Function (BRDF) as the oral landmark of the subject. In some cases, at least part of the color reference target can be covered with a material such that the color reference target has the same optical properties as a color reference target covered in saliva. In some cases, at least one of the three or more color samples can be interchanged manually. In some cases, the color reference target comprises a display. In some cases, the color reference target is made from paper, cardboard, plastic, metal, porcelain, dentin or enamel, or a combination thereof.

In some cases, at least one of the three or more color samples is interchangeable based on the color of the oral landmark of the subject. In some cases, the method described herein further comprises a processing unit, wherein the processing unit performs the processing in (d). In some cases, the processing comprises using the known colors of the three or more color samples based on a color quantification scheme to identify the true colors of the oral landmark in the captured videos or images. In some cases, the adapter is configured to block environmental light from the field of view of the intraoral region of the subject's mouth. In some cases, the oral landmark comprises at least part of a tooth of the subject. In some cases, the oral landmark comprises at least part of an oral tissue of the subject.

In another aspect, provided herein is a method to quantify an average shade or color of one or more teeth of a subject, comprising: identifying the shade or color of at least a part of each of the one or more teeth of a subject by using the method described herein, averaging the shade or color associated with each of the one or more teeth, thereby generating an average shade or color of the one or more teeth of a subject.

As used herein, the term “dental scan” refers to a video or an image frame from a video or an image capture of the intraoral perspective of the teeth arch or of a tooth.

As used herein, the term “color reference target” refers to abject with at least three color samples with known HUE, VALUE and CHROMA according to Munsell Notation, or known Red, Green and Blue values according to sRGB notation and can be used for color calibration.

Unless otherwise defined, all technical and scientific terms used herein have the same meaning as commonly understood by one of ordinary skill in the art to which the methods and systems described herein. Although suitable methods and materials are described below, methods and materials similar or equivalent to those described herein can be used in the practice of the methods and systems described herein. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the methods and systems described herein involves performing or completing selected tasks or steps manually, automatically, or a combination thereof. Moreover, according to actual instrumentation and equipment of preferred embodiments of methods, apparatus and systems described herein, several selected steps could be implemented by hardware or by software on any operating system of any firmware or a combination thereof. For example, as hardware, selected steps could be implemented as a chip or a circuit. As software, selected steps could be implemented as a plurality of software instructions being executed by a computer using any suitable operating system. In any case, selected steps of the method and system described herein could be described as being performed by a data processor, such as a computing platform for executing a plurality of instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

In order to understand the methods and systems described herein and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1A schematically illustrates optical color reference targets, in accordance with some embodiments.

FIG. 1B schematically illustrates an intraoral image captured using the methods and systems provided herein, in accordance with some embodiments.

FIG. 1C schematically illustrates optical color reference targets, in accordance with some embodiments.

FIG. 1D schematically illustrates an intraoral image captured using the methods and systems provided herein, in accordance with some embodiments.

FIGS. 2A-2F schematically illustrate an example intraoral adapter to which a mobile device can be coupled to perform a dental scan, in accordance with some embodiments.

FIG. 3 schematically illustrates a method for conducting a dental scan in, accordance with some embodiments.

FIG. 4 schematically illustrates a computer system that is programmed or otherwise configured to implement methods provided herein.

### DETAILED DESCRIPTION

While various embodiments have been shown and described herein, it will be obvious to those skilled in the art that such embodiments are provided by way of example only. Numerous variations, changes, and substitutions may occur to those skilled in the art without departing from the methods and systems described herein. It should be understood that various alternatives to the embodiments of the methods and systems described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “a,” “an,” and “the,” as used herein, generally refer to singular and plural references unless the context clearly dictates otherwise.

### Overview

The present disclosure provides methods and systems for intraoral imaging with capability for intraoral tissue color or shade identification by using a color reference target to enhance remote dental monitoring capabilities. As used herein, intraoral imaging may refer to the acquisition of one or more intraoral videos and/or intraoral images. The methods and systems disclosed herein may provide a convenient solution and user experience for dental users to capture one or more intraoral videos or images using a mobile device such as a smartphone. The methods and systems disclosed herein may provide dentists and orthodontists with a detailed analysis of the user's dental condition based on one or more dental scans captured remotely by the user.

The methods and systems of the present disclosure may also be used to provide dental users with an intuitive, user-friendly interface for remote dental scanning without requiring assistance from a dentist or a dental assistant. The method and system disclosed herein may permit dentists to view high quality videos or images of a user's teeth so that the dentist can monitor the user's teeth or treatment progress without being physically present and without having to provide personalized or customized instructions for how to acquire the intraoral images or videos.

In an aspect, the present disclosure provides methods and systems for a real color dental scan. The methods and systems of the present disclosure may be implemented using a software application that is configured to enable a dental user to capture videos and/or images of intraoral regions. The software application may be used by a user or a subject (e.g., a dental user) in conjunction with a mobile device to remotely monitor a dental anatomy or a dental condition of the subject. A dental anatomy may comprise one or more dental structures of the user, including one or more tooth structures or dental arches of the subject. A dental anatomy may comprise identify tooth or teeth color. The dental condition may comprise a development, a growth, a movement, an appearance, a condition, a physical arrangement, a position, and/or an orientation of the subject's teeth. In some cases, the dental condition may comprise a functional aspect of the user's teeth, such as how two or more teeth contact each other or how the teeth move over a period of time. In some cases, the dental condition may comprise an accumulation of plaque on the one or more teeth, a change in a color or a structure of the one or more teeth, a change in a color or a structure of a tissue adjacent to the one or more teeth, a change in a color or development of intraoral tissue, identify enamel wear patterns, and/or a presence or lack of presence of one or more cavities.

The dental scan may be used to enable remote dental monitoring. As used herein, remote monitoring may refer to monitoring a dental anatomy or a dental condition of a user that is performed at one or more locations remote from the user. For example, a dentist or a medical specialist may monitor the dental anatomy or dental condition in a first location that is different than a second location where the user is located. The first location and the second location may be separated by a distance spanning at least 1 meter, 1 kilometer, 10 kilometers, 100 kilometers, 1000 kilometers, or more.

The remote monitoring may be performed by assessing a dental anatomy or a dental condition of the subject using one or more dental scan captured by the subject when the user is located remotely from the dentist or a dental office. In some cases, the remote monitoring may be performed in real-time such that a dentist is able to assess the dental anatomy or the dental condition when a subject uses a mobile device to acquire one or more dental scan of one or more intraoral regions in the user's mouth. The remote monitoring may be performed using equipment, hardware, and/or software that is not physically located at a dental office.

The software application for dental scan may be configured to run on a mobile device. The mobile device may comprise a smartphone, a tablet, a laptop, or any suitable device that may be used by a user to capture one or more dental scan. The software application may be installed on a mobile device of a user undergoing a dental treatment or who will be undergoing a dental treatment. The software application may be a user-side software application.

In some cases, the user-side software application may be used in a compatible manner with a practitioner-side software application that is accessible by a caregiver. The user-side software application and the practitioner-side software application may enable real-time communication and sharing of dental scan, or data between one or more users and one or more caregivers. The one or more caregivers may comprise, for example, a dentist, an orthodontist, an oral surgeon, individuals having one or more dental specialties, or a dental staff practitioner.

### Color Reference Target for Intraoral Imaging

In an aspect, the present disclosure provides a color reference target for digital camera comprising at least one replaceable color sample, Wherein the color reference target is used for intraoral tissue imaging and appear on the captured imaging of intraoral tissue by mounting in user intraoral or adapter.

In some embodiments the color reference target has at least three color samples, at least for color samples, at least five color samples, at least six color samples, at least seven color samples, at least eight color samples, at least nine color samples or at least ten color samples.

In some embodiments the color reference target is made from paper, cardboard, plastic, metal, porcelain, dentin or enamel.

In some embodiments the color samples are chosen from Munsell color system.

In some embodiments the color samples can be chosen from the following table.

TABLE 1
Optional color samples

| | sRGB | | | Munsell Notation Hue | | |
|---|---|---|---|---|---|---|
| color | Red | Green | Blue | Value/Chrome | | |
| dark skin | 115 | 82 | 68 | 3 | YR | 3.7/3.2 |
| Light skin | 194 | 150 | 130 | 2.2 | YR | 6.47/4.1 |
| Blue sky | 98 | 122 | 157 | 4.3 | PB | 4.95/5.5 |
| Foliage | 87 | 108 | 67 | 6.7 | GY | 4.2/4.1 |
| Blue flower | 133 | 128 | 177 | 9.7 | PB | 5.47/6.7 |
| Bluish green | 103 | 189 | 170 | 2.5 | BG | 7/6 |
| Orange | 214 | 126 | 44 | 5 | YR | 6/11 |
| Purplish blue | 80 | 91 | 166 | 7.5 | PB | 4/10.7 |
| Moderate red | 193 | 90 | 99 | 2.5 | R | 5/10 |
| Purple | 94 | 60 | 108 | 5 | P | 3/7 |
| Yellow green | 157 | 188 | 64 | 5 | GY | 7.1/9.1 |
| Ornge yellow | 224 | 163 | 46 | 10 | YR | 7/10.5 |
| Blue | 56 | 61 | 150 | 7.5 | PB | 2.9/12.7 |
| green | 70 | 148 | 73 | 0.25 | G | 5.4/8.65 |
| Red | 175 | 54 | 60 | 5 | R | 4/12 |
| yellow | 231 | 199 | 31 | 5 | Y | 8/11.1 |
| Magenta | 187 | 86 | 149 | 2.5 | RP | 5/12 |
| Cyan | 8 | 133 | 161 | 5 | B | 5/8 |

| | | | | | |
|---|---|---|---|---|---|
| White | 243 | 243 | 242 | N | 9.5/ |
| Neutral 8 | 200 | 200 | 200 | N | 8/ |
| Neutral 6.5 | 160 | 160 | 160 | N | 6.5/ |
| Neutral 5 | 122 | 122 | 121 | N | 5/ |
| Neutral 3.5 | 85 | 85 | 85 | N | 3.5/ |
| Black | 52 | 52 | 52 | N | 2/ |

In some embodiments at least part of the color samples is representing color of natural tooth.

TABLE 2
Teeth shade color samples

| | |
|---|---|
| | Munsell Notation Hue |
| Shade | Value/Chrome |

| | | |
|---|---|---|
| A1 | 4.5 | 7.8/1.7 |
| A2 | 2.4 | 7.45/2.6 |
| A3 | 1.3 | 7.4/2.9 |
| A 3.5 | 1.6 | 7.05/3.2 |
| A4 | 1.6 | 6.7/3.1 |
| B1 | 5.1 | 7.75/1.6 |
| B2 | 4.3 | 7.5/2.2 |
| B3 | 2.3 | 7.25/3.2 |
| B4 | 2.4 | 7.0/3.2 |
| C1 | 4.3 | 7.3/1.6 |
| C2 | 2.8 | 6.9/2.3 |
| C3 | 2.6 | 6.7/2.3 |
| C4 | 1.6 | 6.3/2.9 |
| D2 | 3.0 | 7.35/1.8 |
| D3 | 1.8 | 7.1/2.3 |
| D4 | 3.7 | 7.05/2.4 |

In some embodiments at least part of the color samples is correlated to intraoral tissue shades captured at 390 nm-to 450 nm wavelength light exposer or quantitative light-induced fluorescence.

In some embodiments the color samples from table 1 and table 2 are exposed to 390 nm-to 450 nm wavelength light and captured to measure the colors properties under the wavelengths.

In some embodiments at least part of the color samples is correlated to intraoral tissue shades captured at 10 nm-to 400 nm wavelength light exposer or ultraviolet.

In some embodiments the color samples from table 1 and table 2 are exposed to 10 nm-to 400 nm wavelength light and captured to measure the colors properties under the wavelengths.

In some embodiments at least part of the color samples is correlated to intraoral tissue shades captured at 780 nm-to 1,000,000 nm wavelength light exposer or IR.

In some embodiments the color samples from table 1 and table 2 are exposed to 780 nm-to 1,000,000 nm wavelength light and captured to measure the colors properties under the wavelengths.

In some embodiments the color samples are combination of any color samples representing natural color, natural tooth color, intraoral tissue appearance under florescence light, intraoral tissue appearance under UV and intraoral tissue appearance under IR.

In some embodiments at least part of the color samples on the color reference target can change according to the intraoral tissue.

In some embodiments the color reference target color samples have the same Bidirectional Reflectance Distribution Function (BRDF) as an intraoral tissue.

In some embodiments at least part of the color reference target can be covered with a liquid with the same properties as saliva.

In some embodiments the color reference target is a sticker, and the user can select the desire sticker and attached it in the intraoral or on the adapter.

In some embodiments the color samples can be replaced manually.

In some embodiments the user gets a set of color samples and change the color samples on the color reference target as needed.

In some embodiments the color reference target is a screen.

In some preferred embodiments the color reference target is controlled by mobile device, and wherein the color samples kind and order can be selected in accordance to the intraoral tissue subject.

In some embodiments the present disclosure provides a method to indicate the color, shade or condition of an intraoral tissue of a user by using the color reference target according to some embodiment of the present disclosure and a mobile device camera.

In some embodiments the mobile device is a smartphone.

In some embodiments the screen is connected to the smartphone wire or wireless and the smartphone control the kind and order of the color samples.

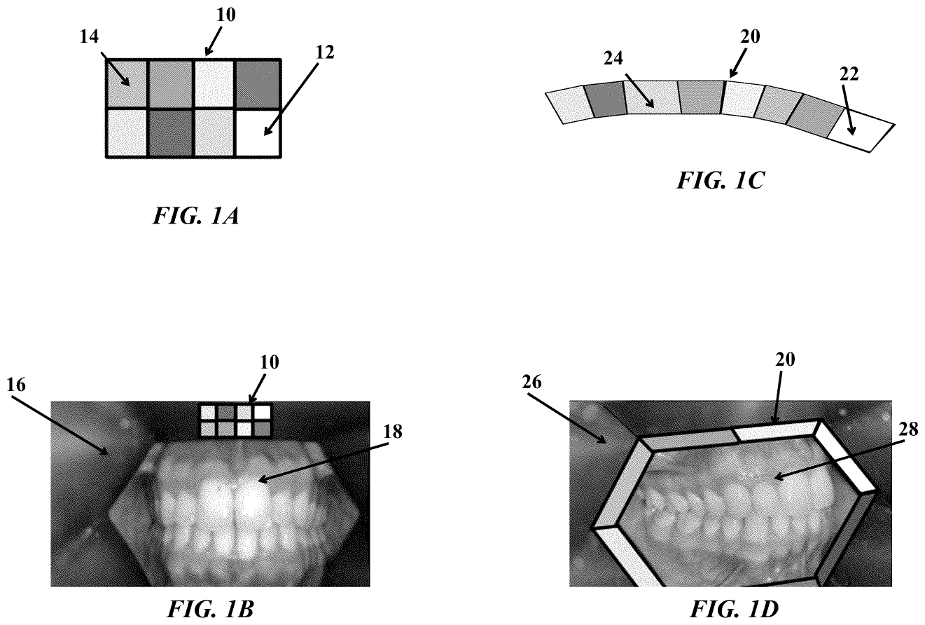

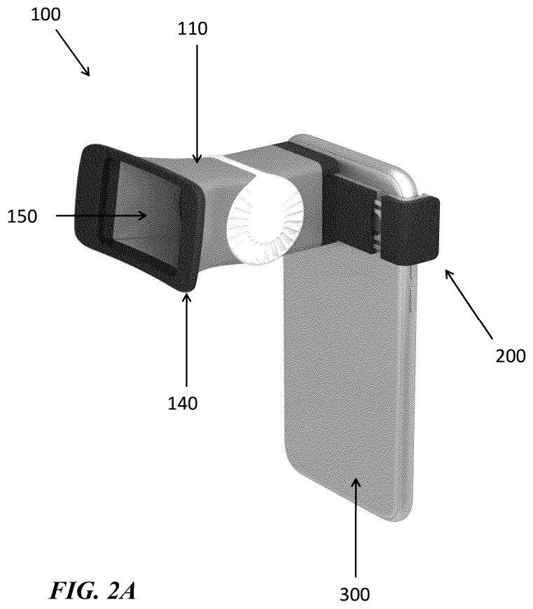

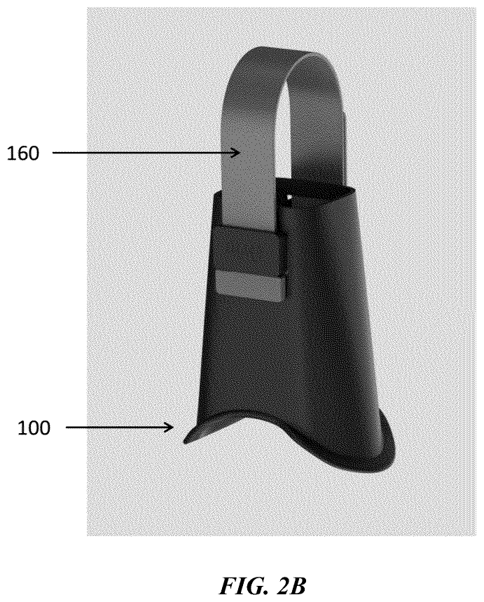

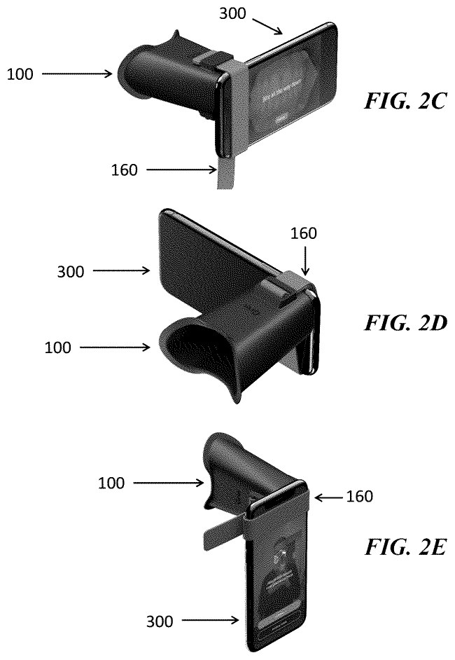

FIG. 1A illustrates an example of color reference target 10 that may be used with the system and methods disclosed herein. The color reference target 10 frame can be made from paper, cardboard, plastic, metal, or any other materials suitable for use as a color sample frame. The color samples, two of which are indicated as 12 and 14, can be made from paper, cardboard, plastic, metal, porcelain, dentin, enamel, or or any other materials suitable for use as a color sample. The size of the color reference target can be chosen to facilitate attachment to an intraoral imaging device. In some cases, a color reference target may be fitted or attached to an intraoral or dental scan adapter. The color reference adapter may be a screen that presents color sample that correspond to the intraoral tissue being imaged. and the desired wavelength exposer. In one example, the color reference target has the following color samples:

TABLE 3
Examples of color samples for an intraoral tissue dental scan

| | sRGB | | | Munsell Notation Hue | | |
|---|---|---|---|---|---|---|
| color | Red | Green | Blue | Value/Chrome | | |
| Light skin | 194 | 150 | 130 | 2.2 | YR | 6.47/4.1 |
| Moderate red | 193 | 90 | 99 | 2.5 | R | 5/10 |
| Orange yellow | 224 | 163 | 46 | 10 | YR | 7/10.5 |
| Red | 175 | 54 | 60 | 5 | R | 4/12 |
| Magenta | 187 | 86 | 149 | 2.5 | RP | 5/12 |

| | | | | | |
|---|---|---|---|---|---|
| White | 243 | 243 | 242 | N | 9.5/ |
| Neutral 6.5 | 160 | 160 | 160 | N | 6.5/ |
| Neutral 3.5 | 85 | 85 | 85 | N | 3.5/ |

In FIG. 1A, the color sample 12 white and color sample 14 moderate red are pointed to for reference.

FIG. 1B illustrates an example view of a color reference target 10 that can be coupled to a dental scan adapter 16 while imaging the intraoral region 18 of a user in accordance with some embodiments of systems and methods described herein.

FIG. 1C illustrates an example of color reference target 20 that may be used with the system and methods disclosed herein. The color reference target frame 20 can be made from paper, cardboard, plastic, or any other suitable material. The color reference target can be folded or deformed to fit into a dental scan adapter as shown in FIG. 1D. In one example, the color reference target has the same color samples as a reference color reference target, like the one shown in FIG. 1A. Additional sets of color samples can be used in the same manner. Color samples may be ordered in any configuration. In FIG. 1C, the color sample 22 white and color sample 24 moderate red are pointed to for reference.

FIG. 1D illustrates an example attachment and view of a color reference target 20 that can be coupled to a dental scan adapter 26 while imaging the intraoral region 28 of a user in accordance with some embodiments of systems and methods described herein.

### Systems and Methods for Identifying the Shade or Color of an Oral Landmark

In another aspect, the present disclosure provides a tooth shade identification system. The tooth shade identification system may comprise an adapter comprising a viewing channel between a proximal portion and a distal portion; a color reference target of at least three color samples; and a mobile device comprising a camera and ability to capture videos or images; wherein a video or an image capture with the mobile device through the adapter viewing channel will include a color reference target.

In some embodiments the mobile device is a smartphone.

In some embodiments the color samples are chosen from Munsell color system.

In some embodiments at least part of the color samples is representing color of natural tooth.

In some embodiments at least part of the color samples is correlated to tooth shades captured at 390 nm-to 450 nm wavelength light exposer or quantitative light-induced fluorescence.

In some embodiments at least part of the color samples is correlated to tooth shades captured at 10 nm-to 400 nm wavelength light exposer or ultraviolet.

In some embodiments at least part of the color samples is correlated to tooth shades captured at 780 nm-to 1,000,000 nm wavelength light exposer or IR.

In some embodiments the color samples are combination of any color samples representing natural color, natural tooth color, tooth appearance under florescence light, tooth appearance under UV and tooth appearance under IR.

In some embodiments the color reference target color samples have the same Bidirectional Reflectance Distribution Function (BRDF) as a tooth.

In some embodiments at least part of the color reference target can be covered with a liquid with the same properties as saliva.

In some embodiments the color samples can be replaced manually.

In some embodiments the color reference target is a screen.

In some preferred embodiments the color reference target is controlled by mobile device, and wherein the color samples kind and order can be selected in accordance to the tooth.

In some embodiments the color reference target is located on the adapter.

In some embodiments the mobile device video or image capture is a color and/or black & white video or image.

In some embodiments the mobile device comprises a processing unit, and wherein the processing unit transforms the shade of at least part of at least tooth according to the color reference target.

In some embodiments the adapter comprises a flange that is sized and shaped to couple the adapter to a user mouth, and wherein the adapter placed in the user mouth blocks environment light.

In some embodiments the system further comprises a light source and wherein the light source can illuminate the viewing channel.

In some preferred embodiments the light source is part of the mobile device.

In some embodiments the adapter can be moved in the user's mouth.

In some embodiments the system is used to identify the shade of at least part of a tooth.

At another aspect, the present disclosure provides a method for tooth shade identification, comprising providing an adapter comprising a viewing channel between a proximal portion and a distal portion and comprising a flange that is sized and shaped to couple the adapter to a user mouth, and wherein the adapter placed in the user mouth blocks environment light;

Providing a mobile device comprising a camera, ability to capture videos or images, processing unit and a light source;
Providing a color reference target;
coupling the mobile device to the adapter and placing the adapter into a user's mouth;
capturing video or an image of at least a part of at least one tooth with the camera of the mobile device through the adapter viewing channel and including the color reference target. processing on the processing unit of the mobile device the video or the image captured and transforming at least a part of at least one tooth according to the color reference target.

In some embodiments the color reference target is located on the adapter.

In some embodiments the color reference target includes at least three colors.

In some embodiments the color samples are chosen from Munsell color system.

In some embodiments at least part of the color samples is representing color of natural tooth.

In some embodiments at least part of the color samples is correlated to tooth shades captured at 390 nm-to 450 nm wavelength light exposer or quantitative light-induced fluorescence.

In some embodiments at least part of the color samples is correlated to tooth shades captured at 10 nm-to 400 nm wavelength light exposer or ultraviolet.

In some embodiments at least part of the color samples is correlated to tooth shades captured at 780 nm-to 1,000,000 nm wavelength light exposer or IR.

In some embodiments the color samples are combination of any color samples representing natural color, natural tooth color, tooth appearance under florescence light, tooth appearance under UV and tooth appearance under IR.

In some embodiments the color reference target color samples have the same Bidirectional Reflectance Distribution Function (BRDF) as a tooth.

In some embodiments at least part of the color reference target can be covered with a liquid with the same properties as saliva.

In some embodiments the color samples can be replaced manually.

In some embodiments the color reference target is a screen.

In some preferred embodiments the color reference target is controlled by mobile device, and wherein the color samples kind and order can be selected in accordance to the tooth.

In some embodiments the processing unit transforms the shade of at least a part of at least tooth according to the reference color target.

In some preferred embodiments the processing includes sampling the color reference target, creating a mapping from the original image colors to colors according to a chosen illuminant, and transforming the original image pixel values according to said mapping.

In some embodiments, the present disclosure provides a method to score user's teeth shade, comprising identification the shade of at least a part of at least two teeth of a user by using some embodiments of the present disclosure, averaging the identification shade results and generating a score or an average shade representing the user's teeth shade.

In some embodiments the system and method describe herein can be used for identifying the color or the shade of a tooth or teeth to select the prefer whitening agent or paste to use to teeth whitening, in addition the system and method describe herein can be use to monitored the color or shade of a tooth or teeth during teeth whitening process.

### Systems and Methods for Intraoral Color or Shade Identification

At another aspect, the present disclosure provides an intraoral tissue shade identifier system, comprising: an adapter comprising a viewing channel between a proximal portion and a distal portion; a color reference target of at least three color samples; and a mobile device comprises a camera and ability to capture videos or images; wherein a video or an image capture with the mobile device through the adapter viewing channel will include a color reference target.

In some embodiments the mobile device is a smartphone.

In some embodiments the color samples are chosen from Munsell color system.

In some embodiments at least part of the color samples is representing color of natural tooth.

In some embodiments at least part of the color samples is correlated to intraoral tissue shades captured at 390 nm-to 450 nm wavelength light exposer or quantitative light-induced fluorescence.

In some embodiments at least part of the color samples is correlated to intraoral tissue shades captured at 10 nm-to 400 nm wavelength light exposer or ultraviolet.

In some embodiments at least part of the color samples is correlated to intraoral tissue shades captured at 780 nm-to 1,000,000 nm wavelength light exposer or IR.

In some embodiments the color samples are combination of any color samples representing natural color, natural tooth color, intraoral tissue appearance under florescence light, intraoral tissue appearance under UV and intraoral tissue appearance under IR.

In some embodiments the color reference target color samples have the same Bidirectional Reflectance Distribution Function (BRDF) as an intraoral tissue.

In some embodiments at least part of the color reference target can be covered with a liquid with the same properties as saliva.

In some embodiments the color samples can be replaced manually.

In some embodiments the color reference target is a screen.

In some embodiments the color reference target is controlled by mobile device, and wherein the color samples kind and order can be selected in accordance to the intraoral tissue subject.

In some embodiments the color reference target is located on the adapter.

In some embodiments the mobile device video or image capture is a color and/or black & white video or image.

In some embodiments the mobile device comprises a processing unit, and wherein the processing unit transforms the shade of at least part of at least tooth according to the color reference target.

In some embodiments the adapter comprises a flange that is sized and shaped to couple the adapter to a user mouth, and wherein the adapter placed in the user mouth blocks environment light.

In some embodiments the system further comprises a light source and wherein the light source can illuminate the viewing channel.

In some preferred embodiments the light source is part of the mobile device.

In some embodiments the adapter can be moved in the user's mouth.

In some embodiments the system is used to identify the shade of at least part of the intraoral tissue.

At another aspect, the present disclosure provides a method for intraoral tissue shade identification, comprising: Providing an adapter comprising a viewing channel between a proximal portion and a distal portion, and comprising a flange that is sized and shaped to couple the adapter to a user mouth, and wherein the adapter placed in the user mouth blocks environment light; Providing a mobile device comprising a camera, ability to capture videos or images, processing unit and a light source; Providing a color reference target; coupling the mobile device to the adapter and placing the adapter into a user's mouth; capturing video or an image of at least a part of at least one tooth with the camera of the mobile device through the adapter viewing channel and including the color reference target; processing on the processing unit of the mobile device the video or the image captured and transforming at least a part of at least one tooth according to the color reference target.

In some embodiments the color reference target is located on the adapter.

In some embodiments the color reference target includes at least three colors.

In some embodiments the color samples are chosen from Munsell color system.

In some embodiments at least part of the color samples is representing color of natural tooth.

In some embodiments at least part of the color samples is correlated to intraoral tissue shades captured at 390 nm-to 450 nm wavelength light exposer or quantitative light-induced fluorescence.

In some embodiments at least part of the color samples is correlated to intraoral tissue shades captured at 10 nm-to 400 nm wavelength light exposer or ultraviolet.

In some embodiments at least part of the color samples is correlated to intraoral tissue shades captured at 780 nm-to 1,000,000 nm wavelength light exposer or IR.

In some embodiments the color samples are combination of any color samples representing natural color, natural tooth color, intraoral tissue appearance under florescence light, intraoral tissue appearance under UV and intraoral tissue appearance under IR.

In some embodiments the color reference target color samples have the same Bidirectional Reflectance Distribution Function (BRDF) as an intraoral tissue.

In some embodiments at least part of the color reference target can be covered with a liquid with the same properties as saliva.

In some embodiments the color samples can be replaced manually.

In some embodiments the color reference target is a screen.

In some preferred embodiments the color reference target is controlled by mobile device, and wherein the color samples kind and order can be selected in accordance to the intraoral tissue subject.

In some embodiments the processing unit transforms the shade of at least a part of an intraoral tissue according to the reference color target.

In some preferred embodiments the processing includes sampling the color reference target, creating a mapping from the original image colors to colors according to a chosen illuminant, and transforming the original image pixel values according to said mapping.

In some embodiments the present disclosure provides a method to score user's intraoral tissue shade, comprising:

identification the shade of at least two parts of an intraoral tissue of a user by using the method according to some embodiments of the present disclosure, averaging the identification shade results and generating a score or an average shade representing the user's intraoral tissue shade.

In some embodiments the system and method describe herein can be used for identifying pathogenic, illness or disorder in a intraoral tissue by identifying abnormal color or shade in intraoral tissue.

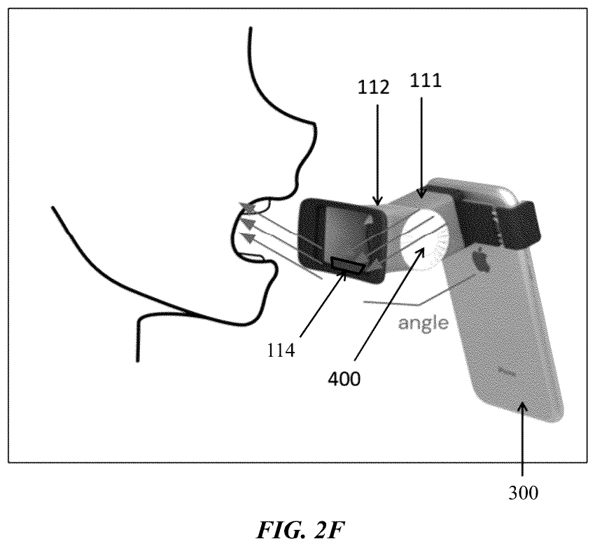

FIG. 2A illustrates an example of an adapter 100 that may be used with the systems and methods disclosed herein. The adapter 100 may comprise an elongated housing 110 with a mounting mechanism 200 and a mobile device 300 coupled to the intraoral adapter 100 via the mounting mechanism 200. The mounting mechanism 200 may be configured to couple the mobile device 300 to the adapter 100 such that a longitudinal axis of a viewing channel 150 of the intraoral adapter 100 is substantially aligned with an optical axis of one or more cameras of the mobile device 300. The mounting mechanism 200 may be configured to mechanically engage with the mobile device 300 or a casing of the mobile device 300. In some embodiments, the mounting mechanism may comprise an elastic band, a clamp, a hook, a magnet, a bracket, or a holder.

The optical axis of the one or more cameras of the mobile device 300 may be aligned with one or more intraoral regions of the subject's mouth when a flange 140 of the elongated housing 110 is positioned between a tooth portion and a gum portion of the subject's mouth. The mobile device may comprise an imaging device (e.g., a camera) that can be configured to capture the one or more intraoral images or videos.

The viewing channel 150 of the elongated housing 110 may be configured to define a field of view of an intraoral region of a subject's mouth. The field of view may be sized and/or shaped to permit one or more cameras of the mobile device 300 to capture one or more videos or images of one or more intraoral regions in a subject's mouth. In some cases, the videos may comprise one or more intraoral images showing a full dental arch of the subject.

The flange 140 may be sized and shaped to couple the adapter to the subject's mouth when the flange 140 is positioned between a gum portion and a tooth portion of the subject's mouth. The adapter 100 may be suspended from the subject's mouth when the flange 140 is positioned between the gum portion and the tooth portion of the subject's mouth. The gum portion and the tooth portion may be in contact with a first side of the flange 140 and a second side of the flange 140 to support a weight of the adapter 100 when the adapter 100 is suspended from the subject's mouth. The flange 140 may be sized and shaped to permit the subject to move the adapter and/or to adjust a position or an orientation of the adapter relative to one or more intraoral regions in the subject's mouth. Adjusting the position or the orientation of the adapter relative to one or more intraoral regions in the subject's mouth may also adjust a position or an orientation of the camera of the mobile device relative to the one or more intraoral regions in the subject's mouth. Adjusting a position or an orientation of the camera of the mobile device relative to the one or more intraoral regions in the subject's mouth may further adjust a relative position and/or a relative orientation of an optical axis of the camera relative to the one or more intraoral regions in the subject's mouth. The flange 140 may remain between the gum portion and the tooth portion of the subject's mouth while the subject moves the adapter around in the subject's mouth. The flange 140 may be sized and shaped to permit the subject to capture one or more intraoral videos or images of a full dental arch of the subject. In any of the embodiments described herein, the flange 140 of the adapter may be configured to be positioned outside the field of view defined by the viewing channel of the adapter.

FIGS. 2B, 2C, 2D, and 2E illustrate another embodiment of an adapter 100 that may be used to capture intraoral videos or images. In some cases, the adapter 100 may comprise an attachment mechanism 160 for coupling a mobile device 300 to the adapter 100. The attachment mechanism 160 may comprise, for example, a strap for securing the mobile device 300 to the adapter 100. The strap may comprise a flexible and/or compliant material, such as silicone. In some cases, the strap may comprise any biocompatible material, or any material that is dishwasher safe. The strap may be adjustable to enable a user to couple various mobile devices having different sizes, shapes, and/or form factors. The adjustability of the strap may provide several advantages, including improved compatibility with different mobile devices having distinct camera configurations, or imaging sensors disposed on different portions or locations on the mobile device.

The method may permit the user to take one or more intraoral images or videos. The intraoral images or videos may be captured while the user is moving the intraoral adapter, or after the user moves the intraoral adapter to a predetermined location.

FIG. 2f, illustrate another embodiment of an adapter 100 that may be used to capture intraoral videos or images. In some cases, the adapter 100 may comprise a flexible element 400 that can connect the two viewing channels 111 and 112. The adapter can comprise color reference target 114. The color reference target 114 can be coupled to the viewing channel at any location, In some cases, the color reference target 114 is coupled to the upper inner side, left inner side, right inner side, or lower inner side of the viewing channel 112. In some cases, the adapter 100 is connected to a smartphone 300. The adapter 100 can be placed in a user mouth.

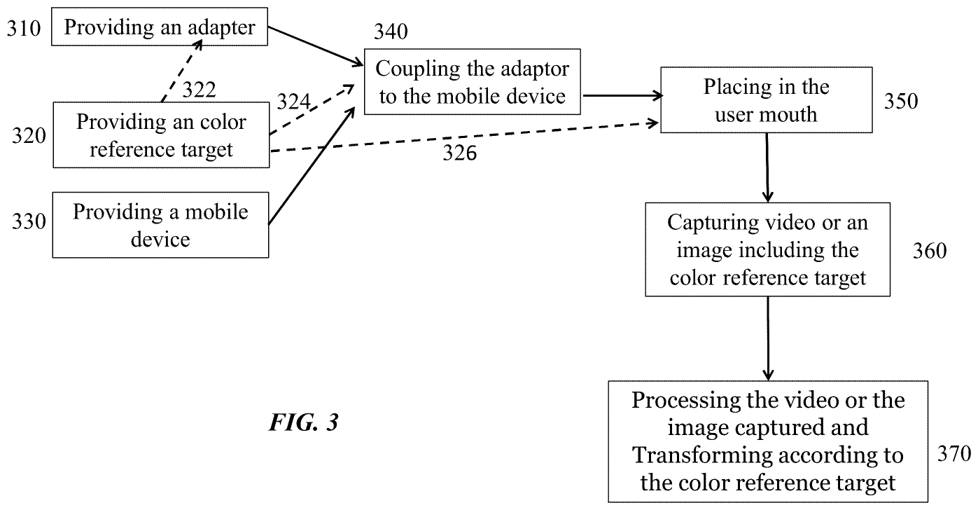

FIG. 3 illustrates an example of a method for dental scan or dental imaging. First, an intraoral adapter 310, a color reference target 320, and a mobile device 330 may be provided to a user. Next, the user may couple the mobile device 330 to an intraoral adapter 310. The color reference target 320 may be attached 322 to the adapter prior to the coupling 340 of the mobile device 330 to the adapter 310. The color reference target 320 may be attached 324 to the adapter prior to the coupling 340 of the mobile device 330 to the adapter 310. Before placing the adapter in the mouth 350 the color reference target may be attached 326 to an intraoral organ such as a tooth. Next, the user may use the mobile device to initiate an intraoral dental scan, a video or an image 360 after placing the adapter in his mouth 350. The dental scan (e.g., videos or images) 370 may be process and transform in the mobile devise according to the color reference target 320.

In some embodiments the methods describe herein can be used for identifying the color or the shade of intraoral tissue. The intraoral tissue can comprise one or more tooth, gums, thong, lips or other intraoral organs.

The dental scan disclosed herein may be used with any type of device or intraoral adapter that is configured to permit capture of a user's teeth or dental structure. The intraoral adapter may be configured to permit the user to capture one or more intraoral videos or images using a mobile device or a smartphone. The intraoral adapter may be configured to position the mobile device or smartphone such that the user is able to capture the images or videos from one or more predetermined positions or viewing angles.

A plurality of frames from an intraoral videos or intraoral images that may be captured using the software application. The intraoral video may comprise a partial or full arch movement video. In an arch movement video, a user may move their head from left to right, right to left, up to down, or down to up to capture a partial or full video of their dental arch. The one or more intraoral videos may be captured while the subject is biting down (i.e., when the subject's upper dental arch and lower dental arch are in contact with or adjacent to each other).

### Computer Systems

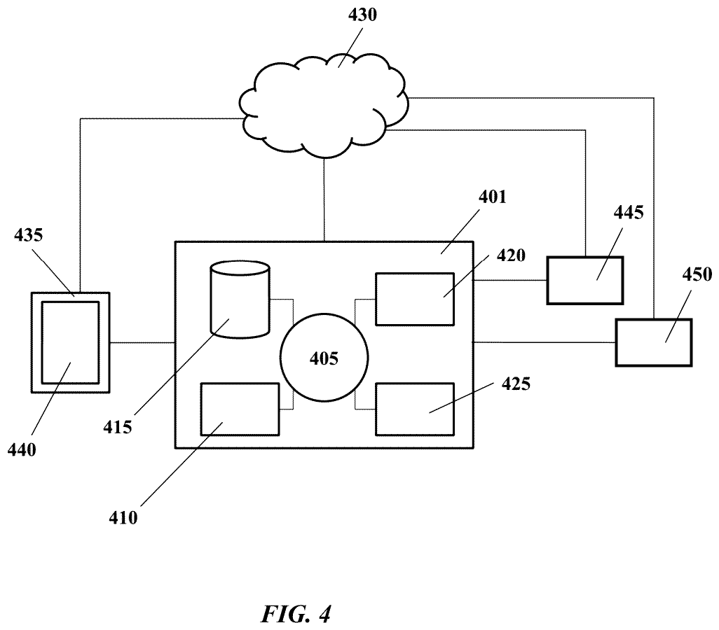

In an aspect, the present disclosure provides computer systems that are programmed or otherwise configured to implement methods of the disclosure. FIG. 4 shows a computer system 401 that is programmed or otherwise configured to implement a method for dental scan. The computer system 401 may be configured to, for example, process intraoral videos or images captured using the camera of the mobile device, and determine the color or shade of at least one of the intraoral tissues during or after the dental scan. The computer system 401 can be an electronic device of a user or a computer system that is remotely located with respect to the electronic device. The electronic device can be a mobile electronic device. The computer system 401 can be a smartphone.

The computer system 401 may include a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk, Solid State drive or equivalent storage unit), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are located external to the computer system 401 (e.g., on a remote server that is in communication with the computer system 401 through an intranet or the Internet).

The computer system 401 can communicate with one or more remote computer systems through the network 430. For instance, the computer system 401 can communicate with a remote computer system of a user (e.g., a subject, a dental user, or a dentist). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a storage unit. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, a portal for a subject or a dental user to view one or more intraoral images or videos captured using a mobile device of the subject or the dental user. In some cases, the electronic display 435 may be the feedback element providing the generated output, for example displaying message or shape or light in accordance to some embodiments. In some cases, the user interface (UI) 440 may be the feedback element providing the generated output, for example generating sound or voice message in accordance to some embodiments. The portal may be provided through an application programming interface (API). A user or entity can also interact with various elements in the portal via the UI. Examples of UI's include, without limitation, a graphical user interface (GUI) and web-based user interface.

The computer system 401 can include or be in communication with a Camera 445 for providing, for example, ability to capture videos or images of the subject or a dental user. And for example, retrieve at least one dental scan date (such as optical object distance) that can be used to analyzed and compered to at least one dental scan properties

The computer system 401 can include or be in communication with a sensor or Sensors 450 including, but not limited to orientation sensor or motion sensor for providing, for example, orientation sensor data or motion sensor data during the dental scan. And for example, retrieve at least one dental scan date (such as acceleration) that can be used to analyzed and compered to at least one dental scan properties

Methods and systems of the present disclosure can be implemented by way of one or more algorithms. An algorithm can be implemented by way of software upon execution by the central processing unit 405. The algorithm can, for example, implement a method for dental scan. The method may comprise processing videos or images captured using the camera of the mobile device or processing dental scan data sensed by at least one sensor that can be used to analyze and compare to at least one dental scan properties and executed to generate output.

While embodiments have been shown and described herein, it will be obvious to those skilled in the art that such embodiments are provided by way of example only. It is not intended that the systems and methods described herein be limited by the specific examples provided within the specification. While the systems and methods described herein has been described with reference to the aforementioned specification, the descriptions and illustrations of the embodiments herein are not meant to be construed in a limiting sense.

Numerous variations, changes, and substitutions will now occur to those skilled in the art without departing from the systems and methods described herein. Furthermore, it shall be understood that all aspects of the systems and methods described herein are not limited to the specific depictions, configurations or relative proportions set forth herein which depend upon a variety of conditions and variables. It should be understood that various alternatives to the embodiments described herein may be employed in practicing the systems and methods described herein. It is therefore contemplated that the systems and methods described herein shall also cover any such alternatives, modifications, variations or equivalents. It is intended that the following claims define the scope of the systems and methods described herein and that methods and structures within the scope of these claims and their equivalents be covered thereby.

What is claimed is:

1. A color reference target for intraoral imaging, comprising one or more interchangeable color samples, wherein the color reference target is configured to be visible when viewed through the viewing channel of an intraoral adapter, wherein the viewing channel of the intraoral adapter is configured to define a field of view of an intraoral region of a subject's mouth.

2. The color reference target of claim 1, wherein the one or more color samples are based on a color quantification scheme.

3. The color reference target of claim 1, wherein the adapter is configured to couple to a camera configured to capture images or videos of the subject's mouth, wherein the captured images or videos comprise an oral landmark of the subject.

4. The color reference target of claim 3, wherein the images or videos are captured under light at a wavelength of 390 nm to 450 nm.

5. The color reference target of claim 1, wherein at least one of the one or more color samples corresponds to a color of a natural tooth, a color of an intraoral tissue under fluorescent light, a color of an intraoral tissue under ultraviolet light, or a color of an intraoral tissue under infrared light, or any combination thereof.

6. A system for identifying the color or shade of an oral landmark of a subject, comprising: an adapter comprising a viewing channel, wherein the viewing channel is configured to define a field of view of an intraoral region of a subject's mouth; and a color reference target comprising three or more color samples, wherein the color reference target appears in the field of view of the subject's mouth.

7. The system of claim 6, wherein the adapter is configured to couple to a camera configured to capture images or videos of an oral landmark of the subject.

8. The system of claim 7, wherein the camera is part of a mobile device.

9. The system of claim 6, wherein the three or more color samples are based on a color quantification scheme.

10. The system of claim 7, wherein the images or videos are captured under light at a wavelength of 390 nm to 450 nm.

11. The system of claim 6, wherein the color reference target is located on the adapter.

12. The system of claim 7, further comprising a processing unit, and wherein the processing unit processes a color of at least part of the oral landmark captured in the images or videos to identify a true color of the at least part of the oral landmark, wherein the processing is based on the three or more color samples of the color reference target.

13. A method for identifying a true color of an oral landmark of a subject, comprising:
(a) providing an adapter comprising a viewing channel configured to define a field of view of an intraoral region of the subject's mouth, wherein the oral landmark is visible in the field of view of the subject's mouth;
(b) providing a color reference target comprising three or more color samples, wherein the color reference target appears in the field of view of the subject's mouth;
(c) capturing videos or images of at least part of the oral landmark through the adapter viewing channel; and
(d) using the color reference target, processing a color of at least part of the oral landmark captured in the videos or images to identify the true color of the at least part of the oral landmark, wherein the processing is based on the three or more color samples of the color reference target.

14. The method of claim 13, wherein the images or videos are captured under light at a wavelength of 390 nm to 450 nm.

15. The method of claim 13, wherein at least one of the three or more color samples corresponds to a color of a natural tooth, a color of an intraoral tissue under fluorescent light, a color of an intraoral tissue under ultraviolet light, or a color of an intraoral tissue under infrared light, or any combination thereof.

16. The method of claim 13, wherein at least one of the three or more color samples can be interchanged manually.

17. The method of claim 13, wherein the color reference target comprises a display.

18. The method of claim 13, further comprising a processing unit, wherein the processing unit performs the processing in (d).

19. The method of claim 13, wherein the processing comprises using the known colors of the three or more color samples based on a color quantification scheme to identify the true colors of the oral landmark in the captured videos or images.

20. A method to quantify an average shade or color of one or more teeth of a subject, comprising: identifying the shade or color of at least a part of each of the one or more teeth of a subject by using the method of claim 13, averaging the shade or color associated with each of the one or more teeth, thereby generating an average shade or color of the one or more teeth of a subject.
